(19) United States
(12) Patent Application Publication
(10) Pub. No.: US 2007/0264202 A1
(43) Pub. Date: Nov. 15, 2007

(54) Pharmaceutical Composition Comprising an Isomer of Betamimetic Agent and an Anti-Cholinergic Agent

(75) Inventors: Amar Lulla, Maharashtra (IN); Geena Malhotra, Maharashtra (IN)

Correspondence Address: CONLEY ROSE, P.C., 5601 GRANITE PARKWAY, SUITE 750, PLANO, TX, 75024, US

(73) Assignee: CIPLA LIMITED, Mumbai (IN)

(21) Appl. No.: 11/574,902

(22) PCT Filed: Sep. 9, 2005
(86) PCT No.: PCT/GB05/03475

§ 371 Date: Jul. 24, 2007

(30) Foreign Application Priority Data
Sep. 9, 2004 (IN) 0970/MUM/2004
Sep. 17, 2004 (IN) 1004/MUM/2004
Oct. 8, 2004 (IN) 1077/MUM/2004
Oct. 11, 2004 (IN) 1088/MUM/2004
Oct. 11, 2004 (IN) 1089/MUM/2004
Jan. 31, 2005 (IN) 0093/MUM/2005
Feb. 28, 2005 (IN) 0222/MUM/2005

(51) Int. Cl. A61K 31/46; A61K 31/537; A61K 9/12; A61M 15/00

(52) U.S. Cl. 424/43; 128/203.12; 514/230.5; 514/304

(57) ABSTRACT

A pharmaceutical composition in a dosage form suitable for inhalation comprises a therapeutically isomer of a betamimetic agent or a salt, solvate, ester, derivative, or polymorph thereof substantially free of the less therapeutically effective isomer(s) of said agent and optionally an anti-cholinergic agent or a salt, solvate, ester, derivative, isomer or polymorph thereof. A preferred composition comprises R-salbutamol sulphate and ipratropium bromide. Methods of making the compositions of the invention are also provided.

The present invention relates to pharmaceutical compositions comprising a betamimetric agent optionally in combination with other active agents, the compositions being useful in the treatment of bronchoconstriction, asthma and related disorders thereof; to methods of preparing the compositions, and to their use in therapy.

Asthma is chronic inflammatory disease affecting about 20 million to 35 million persons worldwide, in which the patient suffers episodes of reversible airways obstruction due to bronchial hyperresponsiveness. Due to inflammation of the bronchial tissues, there is restriction of the bronchial airway leading to bronchoconstriction. Pharmacological intervention is aimed at the prevention and control of asthma symptoms, reducing the frequency and severity of exacerbations, and reversing airflow obstruction. The most commonly administered therapeutic class of drugs is betamimetics, which may be administered either alone or in combination with other related therapeutic agents. Betamimetics are preferably administered by inhalation so as to provide local action and thereby reduce undesired systemic effects. Two main beneficial effects of inhaled betamimetics in asthma are bronchodilation and inhibition of bronchoconstriction induced by exercise and other provocative stimuli. Inhaled short-acting betamimetics like salbutamol (also known as albuterol) and terbutaline are recommended for the relief of acute symptoms, while long-acting agents like salmeterol are used in combination with corticosteroids, anti-cholinergics and leukotriene inhibitors for long-term asthma control and prevent tolerance to the inhaled medication.

One such combination of salbutamol with ipratropium which is available under the trade name Duoneb is marketed by Dey Pharmaceuticals. This contains ipratropium bromide in a concentration of 0.5 mg and albuterol sulphate in a concentration of 3 mg equivalent to albuterol 2.5 mg per 2.5 ml inhalation solution. This is described in U.S. Pat. No. 6,632,842 in which the inhalation solution comprising albuterol and ipratropium is prefilled in one single dispensing container suitable for nebulisation.

Patent application number WO2003013633 to Glaxo Group Limited describes a dry powder pharmaceutical composition comprising a betamimetic and anti-cholinergic agent.

US 2002189610 claims a pharmaceutical formulation comprising a betamimetic agent along with ipratropium wherein the betamimetic agent is formoterol or salmeterol or their salts thereof in a buffered solution suitable for inhalation.

It has been proved that racemic salbutamol, a commonly used bronchodilator, is an exact 50:50 mixture of two enantiomers, the R- and S-isomers. In-vitro studies suggest that the two enantiomers have different binding affinities for the beta-adrenoreceptor, may exert opposing effects on inflammation, demonstrate different effects on mucociliary transport, and display differing pharmacokinetics. The R-isomer has greater bronchodilatory effects than the racemate and may have anti-inflammatory properties. The S-isomer has markedly less affinity for the beta-adrenoreceptor.

Several methods for preparation of levoalbuterol have been described in the prior art such as US patent application number 20040115136 by King Code which describes a method of preparation of levalbuterol tartarate.

Patent application number CN1413976 by Suzhou Junning New Drug Dev CT (CN) describes the synthesis of levosalbutamol and US patent application number US2004054215 to CIPLA Limited discloses a method for obtaining an optically pure R-isomer of albuterol.

Salmeterol is a potent, long lasting betamimetic agent commonly prescribed for the treatment of patients with obstructive airway disease such as asthma. Salmeterol is commonly marketed as a racemate mixture under the trademark SEREVENT.

The R and S isomers of salmeterol are known. European patent application number EP0422889 and U.S. Pat. No. 5,919,827 both relate to the R-isomer of salmeterol and suggest that it has a particularly advantageous profile of action. More particularly, U.S. Pat. No. 5,919,827 suggests that the use of the R-isomer for the treatment of, inter alia, asthma provides a safe and effective therapy while reducing undesirable side effects typically associated with betamimetic agents.

US patent application number 20040136918 claims a combination of R-salmeterol xinafoate and fluticasone propionate as a metered dose aerosol inhalation for the treatment of asthma, chronic obstructive pulmonary disorder, and respiratory tract disorders.

Formoterol has two chiral centers and therefore has possibility of 4 different isomeric combinations of material available. However, it has been found that R,R formoterol is 1000-times more potentially active than its S,S-isomer or any other available isomer. It is well described by, for example, U.S. Pat. No. 6,068,833, U.S. Pat. No. 5,795,564 and U.S. Pat. No. 6,299,863.

Combinations of R,R-formoterol along with corticosteroids in bronchodilating therapy have been described in WO2004047828 which claims a combination of R,R-formoterol and roflumilast; and in US2004019025 which claims a combination of R,R-formoterol and rofleponide.

The present invention hereby provides a pharmaceutical composition comprising a therapeutically effective isomer of a betamimetic agent or a salt, solvate, ester, polymorph or derivative thereof, optionally along with a suitable bronchodilator such as an anti-cholinergic agent or a salt, solvate, ester, isomer, polymorph or derivatives thereof, thereby providing a additive effect.

It is an object of the present invention to provide for a formulation, which provides the advantages of potent and selective therapeutic activity by employing the therapeutically more effective isomer of betamimetic agent or a salt, solvate, ester, polymorph or derivative thereof.

It is another object of the present invention to provide for a formulation, which comprises a combination of the therapeutically more effective isomer of betamimetic agent or a salt, solvate, ester, polymorph or derivative thereof, optionally along with an anti-cholinergic agent or a salt, solvate, ester, isomer, polymorph or derivative thereof, thereby providing additive effect for patients with chronic disorders of the respiratory tract such as asthma and COPD.

It is still another object of the present invention to provide for a formulation, which employs the therapeutically effective isomer of a betamimetic agent or a salt, solvate, ester, polymorph or derivative thereof, thereby providing a more potent formulation and therefore avoiding side effects associated with higher dosages.

Another object of this invention is to provide for a pharmaceutical composition for treatment of respiratory disorders such as asthma, chronic obstructive pulmonary disorder (COPD), and disorders resulting in bronchoconstriction.

It is yet another object of the invention to provide for a method of preparation of the pharmaceutical composition of the invention.

According to the present invention, there is provided a pharmaceutical composition in a dosage form suitable for inhalation, which composition comprises a therapeutically effective isomer of a betamimetic agent or a salt, solvate, ester, derivative or polymorph thereof substantially free of the less therapeutically effective isomer(s) of said agent, and optionally an anti-cholinergic agent or a salt, solvate, ester, derivative, isomer or polymorph thereof.

There are also provided methods for preparing pharmaceutical compositions according to the invention.

There is also provided novel pharmaceutical compositions for the treatment of respiratory and related disorders such as asthma, COPD, and such other disorders, which result in bronchoconstriction.

The invention employs the most active, therapeutically speaking, isomer of a betamimetic agent. Substantially free of the less therapeutically effective isomer(s) means that these isomers will not be present in any significant amount. Suitably, such isomers will be present at no more than 10% w/w of betamimetic, more preferably 1% w/w or less. Thus, for example, compositions containing levosalbutamol, (R)-salmeterol or R,R-formoterol are substantially free of the S-isomers of these compounds.

Betamimetic agents are known to provide a bronchodilator effect to patients by acting on the β-2 adrenergic receptors in the airway smooth muscles and the bronchial smooth muscles, resulting in relief from the symptoms of breathlessness. More particularly, betamimetic agents have been shown to increase the conductance of potassium channels in airway muscle cells, leading to membrane hyperpolarization and relaxation. Therefore being very selective in their activity, they are a preferred class of bronchodilators. This class comprises compounds such as salbutamol, salmeterol, formoterol, rimeterol and acebutolol.

The present invention is advantageous in that it employs the therapeutically effective isomers of these compounds. Compounds such as salbutamol, salmeterol and formoterol are known to exist as their R- and S-isomers and for each of these compounds the R-isomers are more active than the S-isomers. The difference in activity is such that the S-isomer has markedly less affinity for the beta-adrenoreceptors than the R-isomer. The R-isomer has greater bronchodilatory effects and has anti-inflammatory properties. Therefore, the R-isomers are much more therapeutically active, and are hence preferred. Although use of only these compounds helps to bring about sufficient dilation of the bronchial vessels so as to provide relief, in order to avoid development of tolerance to such drugs, it is preferable to give them in combination with other bronchodilators. Such combinations enhance the bronchodilatory activity due to an additive effect. Anticholinergic agents are a preferred class of compounds, and can act additively to provide enhanced activity and avoid any side effects. Anti-cholinergics include compounds such as ipratropium, atropine, tiotropium or salts, solvates, esters, isomers, polymorphs or derivatives thereof. The particular combination of a betamimetic agent and an anticholinergic proves to be highly effective because both the drugs provide bronchodilation by different mechanism of action, which therefore results in an additive effect. These anti-cholinergics act on the muscarinic receptors that are present in the large central airways thus relaxing the central airways. And compounds like levosalbutamol, (R)-salmeterol and R,R-formoterol act on the peripheral airways and relax those muscles. Therefore, the combination provides enhanced activity due to additive effect. The onset of action is much faster due to the use of therapeutically effective isomers such as levosalbutamol, (R)-salmeterol or R,R-formoterol and the duration of activity is longer due to the anticholinergic compounds such as ipratropium and tiotropium. The duration of action gets still prolonged if a longer acting betamimetic such as (R)-salmeterol and R,R-formoterol is used.

Commercially available formulation of racemic salbutamol sulphate and ipratropium bromide by Dey Pharmaceuticals as described in U.S. Pat. No. 6,632,842, claims a combination comprising 2.5 mg of salbutamol sulphate and 500 mcg of ipratropium bromide. However, with the use of the therapeutically effective isomer i.e. levosalbutamol, the dosage of the betamimetic agent to be administered is reduced to half or even less than half. Due to this reduced dosage, there are fewer cardiovascular complications, which are associated with higher doses of bronchodilators. Therefore, the use of such a combination comprising a therapeutically effective isomer and an anti-cholinergic agent results in increased patient compliance.

According to one aspect of the present invention levosalbutamol may be formulated as a solid oral dosage forms e.g. tablet, capsule, extended release granules/tablet etc. These are formulated by techniques known to any person skilled in the art.

Levosalbutamol can be blended with diluents, binders, disintegrants, glidants, lubricant and the resulting mixture compressed.

According to another aspect of the present invention levosalbutamol may be formulated as a liquid. The liquid formulation may comprise one or more suitable ingredients for liquid formulations like thickeners, sweeteners, buffering agents, preservatives, artificial colors, chelating agents/sequestering agents and flavours and other ingredients in addition to levosalbutamol.

A liquid formulation according to the present invention preferably has a pH in the range of 3.0 to 5.0.

In a further aspect of the present invention, there is provided a process for manufacture of a pharmaceutical composition comprising levosalbutamol in a suitable liquid carrier.

The manufacturing process comprises, dissolving preservative, sequestering agent and buffers in specified amount of purified water followed by addition of the drug. This is followed by the addition of other ingredients to the above solution. The pH is checked and finally the volume is made up.

The levosalbutamol according to the present invention can be administered in a dose of 30 mcg to 8 mg.

In the compositions of the invention, preferred ranges for the amount of betamimetric agent and the amount of anticholinergic agent (separately) include 0.005-0.5% w/w and 0.05 to 0.2% w/w. Preferred compositions include from 0.005 to 0.5% w/w levosalbutamol and from 0.005 to 0.5% w/w ipratropium, more preferably from 0.05 to 0.2% w/w levosalbutamol and from 0.05 to 0.2% w/w ipratropium.

Levosalbutamol may, for example, be administered in the doses of 0.63 mcg to 1.5 mg up to 3-4 times daily. Ipratropium bromide can, for example, be administered in a concentration of 100 mcg to 500 mcg, 3-4 times daily. (R)-salmeterol can, for example, be administered up to 8 mg one to four times daily whereas R,R-formoterol can, for example, be administered in doses between 8 mcg to 25 mcg daily. The combination is administered by the inhalation route so as to provide local action and thus avoid undesirable systemic effects.

Specific combinations of any one of R-salbutamol, R-salmeterol, and R, R-formoterol with any one of ipratropium, atropine or tiotropium may be used in any of the inhalation formulations of the invention—for example MDI, DPI or inhalation solution/suspension form.

The combination may further be combined with pharmaceutically acceptable excipients in order to provide a suitable formulation. The combination may, for example, be formulated as an inhalation solution for nebulisation, as an aerosol composition, as dry powder composition for inhalation.

In an aerosol composition, the drugs may be added together or separately in solution or suspension in a propellant. An aerosol formulation according to present invention may optionally comprise in addition to levosalbutamol, ipratropium and at least one propellant, other pharmaceutically acceptable agents such as cosolvents, antioxidants and/or surfactants.

Suitable propellants include hydrocarbons such as n-propane, n-butane or isobutane or mixtures of two or more such hydrocarbons such as monofluorotrichloromethane, dichlorodifluoromethane and halogen-substituted hydrocarbons, for example fluorine-substituted methanes, ethanes, propanes, butanes, cyclopropanes or cyclobutanes, particularly 1,1,1,2-tetrafluoroethane (HFA134a) and 1,1,1,2,3,3,3-heptafluoropropane (HFA227) or mixtures of two or more such halogen-substituted hydrocarbons. Where the active ingredient is present in suspension in the propellant, i.e. where it is present in particulate form dispersed in the propellant, the aerosol composition may also contain a lubricant and a surfactant, which may be chosen from those lubricants and surfactants known in the art.

Other suitable aerosol compositions include surfactant-free or substantially surfactant-free aerosol compositions. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurised metered dose inhalation device. The surfactants may be selected from those known in the art like oils such as corn oil, olive oil, cottonseed oil & sunflower oil, mineral oil like liquid paraffin, oleic acid, phospholipids such as lecithin and citric acid, sorbitan trioleate, glycerol, glycol and the like, in the range of 0.0001-15% by weight with respect to the active.

In a further aspect of the present invention there is provided a process for the manufacture of aerosol composition which comprises I) addition of levosalbutamol & ipratropium to a suitable canister, II) crimping the canister with the metered valve, III) charging with the suitable propellant. The process also optionally comprises dissolution of surfactant in a co-solvent after addition of the drugs.

For dry powder inhalation, the drugs may be used alone or optionally together with a finely divided pharmaceutically acceptable carrier, which is preferably present and may be chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose. The dry powder may be in capsules of gelatin or HPMC, or in blisters or alternatively, the dry powder may be contained as a reservoir in a multi-dose dry powder inhalation device. The particle size of the active ingredient and that of the carrier where present in dry powder compositions, can be reduced to the desired level by conventional methods, for example by grinding in an air-jet mill, ball mill or vibrator mill, microprecipitation, spray-drying, lyophilisation or recrystallisation from supercritical media.

According to the present invention there is also provided a process for manufacture of a dry powder inhaler comprising levosalbutamol and ipratropium, which process comprises mixing the active ingredients optionally with a suitable carrier, and providing the ingredients in a suitable dry powder inhaler.

For inhalation solutions, the drugs may be combined with suitable excipients such as tonicity adjusting agents, pH regulators, chelating agents in a suitable vehicle. The preferred tonicity adjusting agent is sodium chloride. The pH regulators may be selected from pharmacologically acceptable inorganic acids or organic acids or bases. Preferred inorganic acids are selected from the group consisting of hydrochloric acid, hydrobromic acid, nitric acid, sulphuric acid, phosphoric acid and the like. Preferred organic acids are selected from the group consisting of ascorbic acid, citric acid, malic acid, tartaric acid, maleic acid, succinic acid, fumaric acid, acetic acid, formic acid and propionic acid. Preferred inorganic acids are hydrochloric acid & sulphuric acid. For organic acids, ascorbic acid, citric acid and fumaric acid are preferred acids. Preferred inorganic bases are selected from the group consisting of sodium hydroxide, potassium hydroxide, ammonium hydroxide, sodium carbonate, calcium hydroxide. Preferred organic bases are selected from the group consisting of methyl amine, ethyleneimine, hydroquinone, ethyleneimine, ethylamine, dimethylamine, ethanolamine, butylamine, diethylamine. The preferred base is sodium hydroxide. Preferably a nasal inhalation formulation as provided by the present invention has a pH in the range of 3 to 5.

Suitable chelating or complexing agents may be used in the compositions of the present invention, and may be molecules which are capable of entering into complex bonds. Preferable those compounds should have the effect of complexing cations most preferably metal cations, The preferred agent is ethylenediaminetetraacetic acid (EDTA) or a salt thereof, such as the disodium salt.

Liquid vehicles for use in the compositions of the invention (particularly inhalation solutions or suspensions) include, but are not limited to, polar solvents, including, but not limited to, compounds that contain hydroxyl groups or other polar groups. Such solvents include, but are not limited to, water or alcohols, such as ethanol, isopropanol, and glycols including propylene glycol, polyethylene glycol, polypropylene glycol, glycol ether, glycerol and polyoxyethylene alcohols.

Further polar solvents also include protic solvents, including, but not limited to, water, aqueous saline solutions with one or more pharmaceutically acceptable salt(s), alcohols, glycols or a mixture thereof. For a saline solution as the solvent or as a component thereof, particularly suitable salts are those which display no or only negligible pharmacological activity after administration.

An Anti-microbial preservative agent may be added for multi-dose packages. Suitable preservatives will be apparent to the skilled person, particularly benzalkonium chloride or benzoic acid or benzoates such as sodium benzoate, sorbic acid or sorbates such as potassium sorbates in the concentration known from the prior art. Preferably, benzalkonium chloride is added to the formulation.

According to the present invention there is also provided a process for the manufacture of an inhalation solution comprising levosalbutamol and ipratropium The process comprises dissolving the drugs and optionally the, chelating agents, tonicity adjusting agents and any other suitable ingredient in a vehicle and adjusting the pH using a suitable pH adjusting agent.

It will be readily apparent to one skilled in the art that varying substitutions and modifications may be made to the invention disclosed herein without departing from the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention, which is limited only by the claims provided by this document.

The R-isomer of salbutamol sulphate has shown improvement in the Fine Particle Dose (FPD) compared to racemic salbutamol sulphate. The results are as follows:

| | R-salbutamol | | | Racemate |
|---|---|---|---|---|
| | sulphate inhaler | | | salbutamol |
| Fine Particle | Trial I | Trial II | Trial III | sulphate inhaler |
| Dose (%) | 51.38 | 51.90 | 53.22 | 40.80 |

Before testing, both the R-isomer and the racemate are micronised in an identical way.

Cascade analysis gives a value for FPD (Fine particle dose−particles below 4.7 microns)/% FPF (fine particle fraction) which gives a measure of the quantity of particles which have probability to reach the lungs.

The test is done according to USP using Cascade impactor.

A study was conducted to compare bronchodilator responses to levosalbutamol sulphate and racemic salbutamol sulphate administered via metered dose inhaler in a randomized double-blind, single-dose, crossover study. In this study single doses of 100 mcg levosalbutamol sulphate and 200 mcg racemic salbutamol sulphate were administered via MDI in subjects with stable mild to moderate bronchial asthma, who were then monitored over a period of 6 hours. It was found that 100 mcg levosalbutamol sulphate and 200 mcg racemic salbutamol sulphate produced equivalent time-dependant bronchodilator responses over 6 hours.

Thus, it is clear from the study that a reduced dose of levosalbutamol sulphate is required compared to racemic salbutamol sulphate to have the same therapeutic effect.

We have found that levosalbutamol is more free flowing than the racemate and has the advantage of giving better suspension and dispersion characteristics.

The following examples are for illustration but do not limit the scope of the invention.

### EXAMPLE 1

### CFC Inhaler

| Sr. | | |
|---|---|---|
| No. | Ingredients | Qty/can |
| 1. | Levosalbutamol sulphate | 12.00 mg |
| 2. | Lecithin | 0.60 mg |
| 3. | Propellant 11 | 5.70 g |
| 4. | Propellant 12 | 14.70 g |

a) Levosalbutamol sulphate and lecithin were added with propellant 11
(b) The slurry formed was filled in the canisters.
(c) This was crimped with the suitable valve and
(d) Charged with propellant 12 through the valve.

### EXAMPLE 2

### HFA Inhaler

| Sr. | | |
|---|---|---|
| No. | Ingredients | Qty/can |
| 1. | Levosalbutamol sulphate | 12.00 mg |
| 2. | HFA 134a | 18.2 gm |

a) Levosalbutamol sulphate was added to the canister.
b) The canister was crimped with the metered valve and
c) Charged with 1,1,1,2-tetrafluoroethane (HFA134a) and sonicated.

### EXAMPLE 3

### HFA Inhaler

| Sr. | | |
|---|---|---|
| No. | Ingredients | Qty/can |
| 1. | Levosalbutamol sulphate | 12.00 mg |
| 2. | HFA227 | 20.4 gm |

a) Levosalbutamol sulfate was added to the canister.
b) The canister was crimped with the metered valve and
c) Charged with either 1,1,1,2,3,3,3-heptafluoropropane (HFA227) and sonicated

### EXAMPLE 4

### HFA Inhaler

| Sr. | | |
|---|---|---|
| No. | Ingredients | Qty/can |
| 1. | Levosalbutamol sulphate | 12.00 mg |
| 2. | Alcohol | 0.364 gm |
| 3. | Oleic acid | 0.0024 mg |
| 4. | HFA 134a | 17.84 g |

a) Levosalbutamol was added to the canister.
b) Alcohol and surfactant were added to (a) and sonicated.
c) The canister were crimped with the metered valve and
d) Charged with 1,1,1,2-tetrafluoroethane (HFA134a).

### EXAMPLE 5

| Ingredients | Qty/can |
|---|---|
| Levosalbutamol sulfate | 12.00 mg |
| Lactose | 12.00 mg |
| HFA134a | 18.2 gm |

a) Levosalbutamol sulphate was added to the canister.
b) lactose was added to (a)
c) The canister were crimped with the metered valve and
d) Charged with 1,1,1,2-tetrafluoroethane (HFA134a) and sonicated.

### EXAMPLE 6

| Ingredients | Qty/can |
|---|---|
| Levosalbutamol sulfate | 12.00 mg |
| Ethanol | 0.364 gms |
| HFA227 | 20.136 gms |

a) Levosalbutamol was added to the canister.
b) Alcohol was added to (a) and Sonicated
c) The canister were crimped with the metered valve and
d) Charged with HFA227.

### EXAMPLE 7

| Ingredients | Qty/can |
|---|---|
| Levosalbutamol sulfate | 12.00 mg |
| Magnesium stearate | 0.0012 mg |
| HFA227 | 20.5 gms |

a) Levosalbutamol was added to the canister.
b) Magnesium stearate was added to (a)
c) The canister were crimped with the metered valve and
d) Charged with HFA227 and sonicated.

### EXAMPLE 8

| Ingredients | Qty/can |
|---|---|
| Levosalbutamol sulfate | 12.00 mg |
| Isopropyl myristate | 0.0012 mg |
| HFA227 | 20.5.6 gms |

a) Levosalbutamol was added to the canister.
b) Isopropyl myristate added to (a)
c) The canister were crimped with the metered valve and
d) Charged with HFA 227 and sonicated.

### EXAMPLE 9

### HFA Inhaler

| Sr. | | |
|---|---|---|
| No. | Ingredients | Qty/can |
| 1. | Levosalbutamol sulphate | 12.00 mg |
| 2. | Alcohol | 0.364 gm |
| 3. | Oleic acid | 0.0024 mg |
| 4. | HFA227 | 17.84 g |

a) Levosalbutamol was added to the canister.
b) Alcohol and surfactant were added to (a) and Sonicated
c) The canister were crimped with the metered valve and
d) Charged with HFA227.

### EXAMPLE 10

Tablet Formulations

| Sr. | | |
|---|---|---|
| No | Ingredients | Qty (mg/tab) |
| 1. | Levosalbutamol sulphate | 2.40 |
| 2. | Starch | 66.00 |
| 3. | Microcrystalline cellulose | 10.00 |
| 4. | Lactose | 130.00 |
| 5. | Sodium starch glycollate | 10.00 |
| 6. | Starch | 3.40 |
| 7. | Gelatin | 1.40 |
| 8. | Purified water | Qs |
| 9. | Colloidal silicon dioxide | 1.20 |
| 10. | Talc | 2.60 |
| 11. | Magnesium stearate | 3.00 |

Process:

1 and a part of 2 were cosifted to form premix A. 2,3,4,5 were loaded along with premix A into a product bowl. A starch gelatin paste was formed using 6, 7, 8. The starch gelatin paste was sprayed into the blend in the product bowl to from granules. The granules so obtained were lubricated with 9,10,11 and compressed.

### EXAMPLE 11

| Sr. No. | Ingredients | Qty (mg/tab) |
|---|---|---|
| 1. | Levosalbutamol sulphate | 1.20 |
| 2. | Starch | 33.00 |
| 3. | Microcrystalline cellulose | 5.00 |
| 4. | Lactose | 65.00 |
| 5. | Sodium starch glycollate | 5.00 |
| 6. | Starch | 1.70 |
| 7. | Gelatin | 0.70 |
| 8. | Purified water | Qs |
| 9. | Colloidal silicon dioxide | 0.60 |
| 10. | Talc | 1.30 |
| 11. | Magnesium stearate | 1.50 |

Process:

1 and a part of 2 was cosifted to form premix A. 2,3,4,5 were loaded along with premix A into a product bowl. A starch gelatin paste was sprayed using 6, 7, 8. The starch gelatin paste was sprayed into the blend in the product bowl to from granules. The granules so obtained are lubricated with 9, 10, 11 and compressed.

### EXAMPLE 12

| Sr. | | |
|---|---|---|
| No | Ingredients Qty (mg/tab) | |
| 1. | Levosalbutamol Sulfate | 2.00 |
| 2. | Sodium Chloride | 70.00 |
| 3. | Polyethylene oxide | 20.00 |
| 4. | Lactose monohydrate | 75.50 |
| 5. | Hydroxypropyl cellulose | 30.00 |
| 6. | Colloidal silicon dioxide | 1.50 |
| 7. | Magnesium stearate | 1.00 |
| | Film coating | |
| 9. | Cellulose acetate | 6.6 |
| 10 | Hydroxypropylmethyl cellulose | 1.0 |
| 11 | Polyethylene glycol | 0.4 |
| 12. | Ethanol | qs |
| 13. | Methylene chloride | qs |

All the tabletting ingredients except magnesium stearate were sifted. The sifted ingredients were then lubricated using magnesium stearate. The blend so formed was compressed to form tablets. Cellulose acetate, hydroxypropylmethylcellulose and polyethylene glycol were in ethanol and methylene chloride mixture to form a film coating solution. The tablets were then coated with the film coating solution and were drilled on laser drilling machine.

### EXAMPLE 13

| Sr. | | |
|---|---|---|
| No | Ingredients | Qty (mg/tab) |
| 1. | Levosalbutamol sulfate | 2.4 |
| 2. | Hydroxypropylmethyl cellulose | 30.0 |
| 3. | Lactose monohydrate | 63.35 |
| 4. | Talc | 1.5 |
| 5. | Colloidal silicon dioxide | 1.5 |
| 6. | Magnesium stearate | 0.75 |
| 7. | Magnesium stearate | 0.5 |

Levosalbutamol sulfate and lactose were cosifted to form premix I. A blend of HPMC, colloidal silicon dioxide, talc, magnesium stearate and premix I was made. This blend was then subjected to slugging. The tablets so formed were then milled and further passed through appropriate mesh. The granules so obtained were then lubricated with magnesium stearate

### EXAMPLE 14

| Sr. | | |
|---|---|---|
| No | Ingredients | Qty (mg/tab) |
| | Tablets | |
| 1. | Levosalbutamol Sulfate | 2.00 |
| 2. | Calcium sulfate | 20.00 |
| 3. | Croscarmellose sodium | 10.00 |
| 4. | Lactose monohydrate | 76.50 |
| 5. | Colloidal silicon dioxide | 1.50 |
| 6. | Ethanol | qs |
| 7. | Magnesium stearate | 1.00 |
| | Film coating | |
| 9. | Ethyl cellulose | 4.2 |
| 10 | Hydroxypropylmethyl cellulose | 3.4 |
| 11 | Polyethylene glycol | 0.4 |
| 12. | Ethanol | qs |
| 13. | Methylene chloride | qs |

Levosalbutamol sulfate, calcium sulfate, lactose monohydrate, croscarmellose sodium, and colloidal silicon dioxide were sifted to form premix A. the premix A was granulated using ethanol. The granules so formed were lubricated using magnesium stearate and compressed to form tablets. Ethyl cellulose, hydroxypropylmethylcellulose and polyethylene glycol were in ethanol and methylene chloride mixture to form a film coating solution. The tablets were then coated with the film coating solution and were drilled on laser drilling machine.

### EXAMPLE 15

### Liquid

| Sr. No | Ingredients | Qty (% w/v) |
|---|---|---|
| 1. | Levosalbutamol sulphate | 0.0241 |
| 2. | Sodium benzoate | 0.200 |
| 3. | Hydroxy propyl methylcellulose | 0.300 |
| 4. | Disodium edetate | 0.050 |
| 5. | Sodium citrate | 0.100 |
| 6. | Citric acid monohydrate | 0.200 |
| 7. | Sodium chloride | 0.100 |
| 8. | Sweet orange | 0.200 |
| 9. | Sodium saccharin | 0.100 |
| 10. | Sunset yellow | 0.004 |
| 11. | Purified water | q.s. 100.00 |

Procedure: In specified amount of purified water was added and ingredients 2, 4, 5 and 6 were dissolved. 1 was added to the above solution followed by ingredient 9, 7 and 3. The pH was adjusted between 3.0 to 5.0. Ingredient 8 and 10 were added and the volume was made up using 11 and mix for specified time.

### EXAMPLE 16

### Liquid

| Sr. No | Ingredients | Qty (% w/v) |
|---|---|---|
| 1. | Levosalbutamol sulphate | 0.0241 |
| 2. | Sodium benzoate | 0.200 |
| 3. | Hydroxy propyl methylcellulose | 0.300 |
| 4. | Disodium edetate | 0.050 |
| 5. | Sodium citrate | 0.100 |
| 6. | Citric acid monohydrate | 0.200 |
| 8. | Sweet orange | 0.200 |
| 9. | Sodium saccharin | 0.100 |
| 10. | Sunset yellow | 0.004 |
| 11. | Purified water | q.s. 100.00 |

Procedure: In specified amount of purified water was added and ingredients 2, 4, 5 and 6 were dissolved. 1 l was added to the above solution followed by ingredient 9 and 3. The pH was adjusted between 3.0 to 5.0. ingredient 8 and 10 were and the volume was made up using 11 and mix for specified time.

### EXAMPLE 17

### Liquid

| Sr. No | Ingredients | Qty (% w/v) |
|---|---|---|
| 1. | Levosalbutamol sulphate | 0.0241 |
| 2. | Sodium benzoate | 0.200 |
| 3. | Sorbitol solution 70% | 40.00 |
| 4. | Disodium edetate | 0.050 |
| 5. | Sodium citrate | 0.100 |
| 6. | Citric acid monohydrate | 0.200 |
| 7. | Sodium chloride | 0.100 |
| 8. | Sweet orange | 0.200 |
| 9. | Sodium saccharin | 0.100 |
| 10. | Sunset yellow | 0.004 |
| 11. | Purified water | q.s. 100.00 |

Procedure: In specified amount of purified water was added and ingredients 2, 4, 5 and 6 were dissolved. 1 was added to the above solution followed by ingredient 9, 7 and 3. the pH was adjust between 3.0 to 5.0. ingredient 8 and 10 were add and the volume was made up using 11 and mix for specified time.

### EXAMPLE 18

### Liquid

| Sr. No | Ingredients | Qty (% w/v) |
|---|---|---|
| 1. | Levosalbutamol sulphate | 0.0241 |
| 2. | Sodium benzoate | 0.200 |
| 3. | Hydroxy propyl methylcellulose | 0.300 |
| 4. | Disodium edetate | 0.050 |
| 5. | Sodium citrate | 0.100 |
| 6. | Citric acid monohydrate | 0.200 |
| 7. | Sodium chloride | 0.100 |
| 8. | Sweet orange | 0.200 |
| 9. | Sucrose | 50.00 |
| 10. | Sunset yellow | 0.004 |
| 11. | Purified water | q.s. 100.00 |

Procedure: In specified amount of purified water was add and ingredients 2, 4, 5 and 6 were dissolved. 1 was added to the above solution followed by ingredient 9, 7 and 3. The pH was adjust between 3.0 to 5.0. Ingredient 8 and 10 were add and the volume was made up using 11 and mix for specified time.

### EXAMPLE 19

| Sr. | | |
|---|---|---|
| No | Ingredients | Qty (% w/v) |
| 1. | Levosalbutamol sulfate | 0.0602 |
| 2. | Sodium chloride | 0.900 |
| 3. | Disodium Edetate | 0.050 |
| 4. | Sulfuric acid | Qs to pH 3.0 to |
| | | 5.0 |
| 5. | Purified water | Qs 100 ml |

The disodium edetate, sodium chloride, levosalbutamol sulfate were dissolved in water and the pH was adjusted.

### EXAMPLE 20

| Sr. | | |
|---|---|---|
| No | Ingredients | Qty (% w/v) |
| 1. | Levosalbutamol sulfate | 0.015 |
| 2. | Sodium chloride | 0.900 |
| 3. | Disodium Edetate | 0.050 |
| 4. | Sulfuric acid | Qs to pH 3.0 to 5.0 |
| 5. | Purified water | Qs 100 ml |

The disodium edetate, sodium chloride, levosalbutamol sulfate were dissolved in water and the pH was adjusted.

### EXAMPLE 21

| Sr. | | |
|---|---|---|
| No | Ingredients | Qty (% w/v) |
| 1. | Levosalbutamol sulfate | 0.0304 |
| 2. | Sodium chloride | 0.900 |
| 3. | Disodium Edetate | 0.050 |
| 4. | Sulfuric acid | Qs to pH 3.0 to 5.0 |
| 5. | Purified water | Qs 100 ml |

The disodium edetate, sodium chloride, levosalbutamol sulfate were dissolved in water and the pH was adjusted.

### EXAMPLE 22

| Sr. | | |
|---|---|---|
| No | Ingredients | Qty mg/cap) |
| 1. | Levosalbutamol sulfate | 1.2 |
| 2. | Lactose | 45.0 |
| 3. | Starch | 20.0 |
| 4. | Microcrystalline cellulose | 33.3 |
| 5. | Magnesium stearate | 0.5 |

All the ingredients were blended and filled in appropriate size capsules.

### EXAMPLE 23

| Sr. No. | Ingredients | Quantity (% w/w). |
|---|---|---|
| 1 | Ipratropium bromide | 0.021 |
| 2 | Levosalbutamol sulphate | 0.060 |
| 3 | Sodium chloride | 0.900 |
| 4 | Sulphuric acid | q.s. |
| 5 | Disodium edetate | 0.05 |
| 6 | Purified water | q.s.to 100 ml. |

Process:
1. Add and dissolve disodium edetate and sodium chloride in freshly boiled and cooled water.
2. Add and dissolve ipratropium bromide and levosalbutamol sulphate in the above solution.
3. Adjust the pH of the solution, if necessary, with the aid of sulphuric acid and make up the volume to 100 ml.

### EXAMPLE 24

| Sr. No. | Ingredients | Mcg/spray |
|---|---|---|
| 1 | Ipratropium bromide | 20 |
| 2 | Levosalbutamol sulphate | 50 |
| 3 | Lecithin | 100% of drug. |
| 4 | Propellant P₁₁ | q.s. |
| 5 | Propellant P₁₂ | q.s. |

Process:
1. Levosalbutamol and Ipratropium bromide are weighed in an aluminium can.
2. Lecithin is dissolved in a sufficient quantity of propellant P₁₁ and added to the aluminium can of step 1.
3. The aluminum can is crimped and sealed.
4. Propellant P₁₂ is then charged through the aluminium can.

### EXAMPLE 25

| Sr. No. | Ingredients | Mcg/spray |
|---|---|---|
| 1 | Ipratropium bromide | 20 |
| 2 | Levosalbutamol sulphate | 50 |
| 3. | Propellant 134a | q.s. |

Process:
1. Levosalbutamol sulphate and ipratropium bromide are weighed in an aluminium can.
2. The can is then crimped and sealed.
3. Propellant P134a are added to make up the required quantity.

### EXAMPLE 26

| Sr. No. | Ingredients | Mcg/spray |
|---|---|---|
| 1 | Ipratropium bromide | 20 |
| 2 | Levosalbutamol sulphate | 50 |
| 3. | Lactose | 300% of the drug |
| 4. | Propellant P227 | q.s. |

Process:
1. Levosalbutamol sulphate and ipratropium bromide are weighed in an aluminium can.
2. Lactose is added to step 1.
3. The can is then crimped and sealed.
4. The can is then filled with Propellant P227.

### EXAMPLE 27

| Sr. No. | Ingredients | Mg/Cap |
|---|---|---|
| 1 | Ipratropium bromide | 0.042 |
| 2 | Levosalbutamol sulphate | 0.100 |
| 3. | Lactose | 24.858 |

Process:
Levosalbutamol sulphate and ipratropium bromide are blended together with Lactose & filled in capsules.

1. A pharmaceutical composition in a dosage form suitable for inhalation, which composition comprises a therapeutically effective isomer of a betamimetic agent or a salt, solvate, ester, derivative, or polymorph thereof substantially free of the less therapeutically effective isomer(s) of said agent wherein the betamimetic agent is R-salbutamol, or R-salmeterol, and an anti-cholinergic agent or a salt, solvate, ester, derivative, isomer or polymorph thereof wherein the anti-cholinergic agent is ipratropium bromide, tiotropium, or atropine; or wherein the betamimetic agent is R,R-formoterol and the anti-cholinergic agent is atropine.

2. The pharmaceutical composition according to claim 1, wherein the therapeutically effective isomer is the R-isomer of the betamimetic agent.

3. The pharmaceutical composition according to claim 1, wherein the betamimetic agent is R-salbutamol sulphate.

4. The pharmaceutical composition according to claim 1, wherein the anti-cholinergic agent is ipratropium bromide.

5. The pharmaceutical composition as claimed in claim 1, comprising suitable pharmaceutically acceptable excipients to form an inhalation formulation.

6. A metered dose inhaler comprising the pharmaceutical composition according to claim 5.

7. The metered dose inhaler comprising a pharmaceutical composition according to claim 6, the composition comprising pharmaceutically acceptable excipients suitable to form a composition for a metered dose inhaler.

8. The pharmaceutical composition according to claim 5, wherein the composition comprises R-salbutamol sulphate, ipratropium bromide, one or more hydrofluorocarbon propellants, and optionally one or more surfactants, or one or more cosolvents and/or one or more antioxidants.

9. A dry powder inhaler comprising the pharmaceutical composition according to claim 5.

10. The dry powder inhaler comprising a pharmaceutical composition according to claim 9, the composition comprising pharmaceutically acceptable excipients suitable to form a composition for a dry powder inhaler.

11. The pharmaceutical composition according to claim 5, wherein the composition comprises R-salbutamol sulphate, ipratropium bromide and a finely divided pharmaceutically acceptable carrier.

12. The pharmaceutical composition according to claim 5, in the form of an inhalation solution/suspension.

13. The pharmaceutical composition according to claim 12, comprising pharmaceutically acceptable excipients suitable to form an inhalation solution or suspension.

14. The pharmaceutical composition according to claim 12 comprising R-salbutamol sulphate, ipratropium bromide, a polar solvent, a tonicity-adjusting agent, an acid, and optionally a chelating agent.

15. A process for preparing the metered dose inhaler according to claim 6, which process comprises adding the active ingredients to a suitable canister, crimping the canister with a metered dose valve, and charging the canister with propellant.

16. A process for preparing the dry powder inhaler according to claim 9, which process comprises mixing the active ingredients optionally with a suitable carrier, and providing the composition in a dry powder inhaler.

17. A process for preparing the pharmaceutical composition according to claim 12, which process comprises dissolving or suspending the active ingredients optionally together with chelating agents, tonicity adjusting agents and any other suitable excipients, in a liquid vehicle, and adjusting the pH.

18. The pharmaceutical composition according to claim 1 for the treatment of respiratory disorders, including asthma, COPD and other disorders resulting in bronchoconstriction.

19. A method comprising using the pharmaceutical composition according to claim 1 in the manufacture of a medicament for treating respiratory disorders, including asthma, COPD and other disorders resulting in bronchoconstriction.

20. The pharmaceutical composition according to claim 1, which is a tablet or oral liquid.
